(19) United States
(12) Patent Application Publication
(10) Pub. No.: US 2008/0214618 A1
(43) Pub. Date: Sep. 4, 2008

(54) Gastric Therapies Amd Compositions Therefor

(75) Inventors: Anthony Morris Roberton, Auckland (NZ); Charmian Jocelyn O'Connor, Auckland (NZ); Iain Gregory Martin, Auckland (NZ); Raid Ghassan Alany, Grafton (NZ)

Correspondence Address: VENABLE LLP, P.O. BOX 34385, WASHINGTON, DC, 20043-9998, US

(73) Assignee: Auckland Uniservices Limited, Auckland (NZ)

(21) Appl. No.: 11/660,944

(22) PCT Filed: Aug. 23, 2005
(86) PCT No.: PCT/NZ05/00223

§ 371 Date: Sep. 10, 2007

(30) Foreign Application Priority Data
Aug. 23, 2004 (NZ) 534887

(51) Int. Cl. A61K 31/56; A61P 1/04

(52) U.S. Cl. 514/338

(57) ABSTRACT

1. A composition, in one or more parts, for the treatment of gastric and/or duodenal H. pylori infections, the composition including effective amounts of: (a) a proton pump inhibitor in a systemically available dosage form; (b) a mucolytic preparation; (c) a proton pump inhibitor in a gastrically available dosage form; and (d) a fatty acid and/or monoglyceride.

### TECHNICAL FIELD

The invention is directed to the treatment of gastric and/or duodenal Helicobacter pylori infections. In particular the invention is directed to a composition for use in such a treatment.

### BACKGROUND TO THE INVENTION

The discovery in the 1980s of infection of the stomach lining with the bacterium Helicobacter pylori was revolutionary. H. pylori has been shown subsequently to be the cause of more than 90% of duodenal ulcers, the majority of gastric ulcers, and is an important causal factor of gastric carcinoma. There is also evidence to suggest that H. pylori colonisation in the stomach could be involved in the aetiology of a variety of non-gastrointestinal conditions.

At present, eradication of H. pylori from the stomach relies on a combination therapy, usually a proton pump inhibitor (for acid suppression) and two or more types of antibiotic. Increasing antibiotic resistance has made treating the infection significantly more difficult. In addition to this, there are issues of patient compliance with the treatment regimen. The standard triple therapy requires use of the drugs by the patient for one to two weeks which raises further issues of compliance and tolerability.

The antibiotics used in conventional triple therapy have to be absorbed in the intestine and then secreted from stomach tissue after blood circulation in order to reach the site of H. pylori colonisation. This will result in drug dilution. The stomach mucus layer prevents them from reaching the H. pylori colonisation site from the direction of the stomach lumen.

### OBJECT OF THE INVENTION

It is an object of the present invention to provide an alternative treatment for gastric and/or duodenal H. pylori infection.

Other objects of the invention will become apparent from the following description.

### SUMMARY OF THE INVENTION

The invention provides a composition, in one or more parts, for the treatment of gastric and/or duodenal H. pylori infections, the composition including effective amounts of:
(a) a proton pump inhibitor in a systemically available dosage form;
(b) a mucolytic preparation;
(c) a proton pump inhibitor in a gastrically available dosage form; and
(d) a fatty acid and/or monoglyceride.

Preferably the fatty acid (“FA”) or monoglyceride (“MG”) has a “minimum bactericidal concentration” (“MBC”) of 5 mM or less against H. pylori.

Preferably the FA or MG is selected from any one or more of; capric acid, lauric acid, myristic acid, myristoleic acid, palmitoleic acid, linolenic acid, monolaurin or monomyristin.

Preferably the FA and/or MG are in a form capable of dispersing or dissolving in the gastric aqueous phase.

Preferably the FA is provided in a salt form.

Preferably the FA and/or MG is combined with a solubilising agent.

Preferably the solubilising agent is a non-ionic surfactant and (d) is a fatty acid.

Preferably the non-ionic surfactant is sorbitan-based surfactant (eg Tween 20, Tween 80), a bile salt surfactant, a Brij surfactant, or a Triton surfactant.

Preferably the non-ionic surfactant is combined with a density modifier and a viscosity enhancer.

Preferably the non-ionic surfactant is Tween 20 or Tween 80.

In an alternative form, the composition preferably includes both an MG and a FA, and the MG assists in emulsifying the FA.

Preferably the MG/FA mixture also includes a solubilising agent.

Preferably the proton pump inhibitor is selected from one or more of omeprazole, lansoprazole, esomeprozole, timoprazole or picoprazole.

Preferably the systemically available dosage form of the proton pump inhibitor is a liquid, semisolid or solid dosage form.

Preferably the systemically available dosage form of the proton pump inhibitor is an enterically coated dosage form.

Preferably the gastrically available proton pump inhibitor dosage form is a liquid, tablet or like dosage form.

Preferably the gastrically available and systemically available proton pump inhibitor dosage forms are the same but are not an enterically coated dosage form.

Preferably the mucolytic agent is selected from a suitable sulfhydryl reagent.

Preferably the mucolytic agent is N-acetylcysteine.

In another aspect the invention provides a kit for the treatment of H. pylori infections, the kit including the components (a) (b) (c) and (d) above.

Preferably the kit also includes a surfactant.

Preferably component (d) and the surfactant are provided together in combination with a density modifier and a viscosity enhancer.

Preferably the composition or individual components of the kit may further include additives, fillers, sweeteners and like compounds.

Preferably the composition or kit further includes at least one antibiotic.

Preferably the components (c) and (d) in the first aspect of the invention are contained in a single dosage form.

Preferably the solubilising agent is included in the single dosage form with component (d).

The invention also provides a method for treating gastric and duodenal H. pylori infections including the steps of sequentially:
(a) administering a proton pump inhibitor in a systemically available dosage form;
(b) administering a mucolytic preparation;
(c) administering a proton pump inhibitor in a gastrically available form, and a fatty acid and/or a monoglyceride together or sequentially in either order; and
(d) optionally, repeating steps (b) and (c) for a time effective to treat the gastric H. pylori infection.

Preferably steps (b) and (c) and (d) occur after fasting.

Preferably step (a) is single, unrepeated, administration step.

Preferably step (a) occurs before fasting.

Preferably step (b) occurs from about 15 minutes to about 3 hours before step (c).

Preferably step (c) is achieved using a single dosage form.

Preferably, the FA and/or MG are in a form capable of dispersing or dissolving in the gastric aqueous phase.

Preferably the FA or MG is selected from any one or more of; capric acid, lauric acid, myristic acid, myristoleic acid, palmitoleic acid, linolenic acid, monolaurin or monomyristin.

Preferably the FA is sodium laurate.

Preferably step (c) includes the administration of a solubilising agent such as a non-ionic surfactant that will form mixed micelles or microemulsions/emulsions with the FA and/or MG.

Preferably the non-ionic surfactant is combined with a fatty acid, a density modifier and a viscosity enhancer.

In broad terms the invention may also be seen to include the use of a proton pump inhibitor in a systemically available dosage form; a mucolytic preparation; a proton pump inhibitor in a gastrically and/or duodenally available dosage form; and a fatty acid and/or a monoglyceride in the manufacture of a pharmaceutical composition for use in the treatment of gastric and duodenal H. pylori infections.

The invention also comprises the use of the composition, kit or method above-described in the management of any disease state caused by or believed to be caused by a gastric or duodenal H. pylori infection.

The invention also provides a composition, in one or more parts, for the treatment of gastric and/or duodenal H. pylori infections, the composition including effective amounts of
(a) a proton pump inhibitor in a systemically available dosage form;
(b) a mucolytic preparation;
(c) a proton pump inhibitor in a gastrically available dosage form; and
(d) a fatty acid and/or monoglyceride together with a non-ionic surfactant.

Preferably component (d) further includes a density modifier and a viscosity enhancer.

### BRIEF DESCRIPTION OF THE DRAWINGS

FIG. 1 The survival of H. pylori in the presence of: FA C12:0 only (▴); FA C12:0 plus 100 μg/mL lansoprazole (); FA C12:0 plus 200 μg/mL lansoprazole (▪); FA C12:0 plus 300 μg/mL lansoprazole (▾); FA C12:0 plus 400 μg/mL lansoprazole (♦); FA C12:0 plus 0.05% (w/w) Tween 20 (Δ); FA C12:0 plus 100 μg/mL lansoprazole plus 0.05% (w/w) Tween 20 (◯); FA C12:0 plus 200 μg/mL lansoprazole plus 0.05% (w/w) Tween 20 (□). Cells were incubated at pH 7.4 for 40 min at 37° C.

FIG. 2 The effect of lansoprazole on the survival of H. pylori in the presence of 0.05% Tween 20 (▴), and in its absence (Δ). Cells were incubated at pH 7.4 for 40 min at 37° C.

FIG. 3 Diagram of apparatus used to model the mucus layer in digestive tract. This test chamber was designed as an in vitro model to investigate the barrier effect of mucus in protecting H. pylori from agents present in the lumen of the stomach. It shows the mucus/H. pylori layer held in position by a stainless steel grid, immersed in a aqueous mixture of Iso-sensitest broth and test additives at pH 7.0. The mucus layer (C2), contains H. pylori, with numbers of colony forming units as specified. It is held as a 1 mm thick disc between the test chamber base (B and C4) and a stainless steel mesh (A and C1). This was then inserted into a Falcon tube containing broth (D).
A. Screw top lid with a recessed top containing a stainless steel grid.
B. Base with recessed well 1 mm deep×10 mm diameter that contains mucus (shaded area—see C2)
C. Cross sectional view of chamber (A & B). 1—stainless steel mesh, 2=mucus, 3=screw top lid, 4=base
D. Chamber in a Falcon tube covered with Iso-sensitest broth (with or without additives).

FIG. 4 The effect of monolaurin (MG C12) on survival of H. pylori in the absence (squares) and presence (diamonds) of Tween 80 (0.4%, w/v), after 40 min exposure at 37° C. and initial pH 7.4.

### DETAILED DESCRIPTION OF THE INVENTION

The invention is generally directed to the treatment of infections of the stomach lining associated with the bacterium Helicobacter pylori present in the gastric/duodenal tract.

The difficulty with treating gastric and duodenal H. pylori infections, is that the H. pylori lives beneath the mucus layer, next to the stomach mucosal cells. The mucus layer protects the stomach surface from acid and pepsin (proteolytic) damage and will additionally serve to protect the H. pylori from attack.

One of the major difficulties in treating gastric H. pylori infections, is the effect that the mucus layer in the stomach has in protecting the H. pylori from direct attack from agents present in the stomach lumen.

The present invention provides a method of treatment and a combination of components which can assist in providing a treatment for H. pylori infections in the stomach. Thus the invention may also be seen to be a method of treating conditions that stem, or are perceived to stem, from such infections (eg duodenal/gastric ulcer, gastric carcinoma). As the use of conventional antibiotics in this treatment is optional only, the issue of resistance with such drugs (such as amoxycillin; erythromycin; metronidazole) is also overcome. Resistance to fatty acids/MGs or fatty acids/MGs+surfactants is unlikely to develop, as the killing appears to be via a physical phenomenon.

In its preferred form, the composition will in broad terms combine a fatty acid (“FA”) or a monoglyceride (“MG”), a proton pump inhibitor and a mucolytic agent. The FA and/or MG should preferably be administered in a form, or using a method, which maximises its ability to be dispersed/dissolved in the aqueous phase of the gastric contents, to facilitate its action against H. pylori in this environment. The FA should preferably be administered as a salt (eg lauric acid may be administered as sodium laurate). The FA/MG could be filled into a hard gelatine capsule or by formulating the fatty acid and/or monoglyceride with a solubilising agent, such as a non-ionic surfactant, that can form mixed micelles or microemulsions/emulsions with the FA/MG. For example, the surfactant could form an oil-in-water emulsion in which the fatty acid and/or monoglyceride is finely and uniformly distributed so that a higher concentration of FA/MG is retained in the aqueous phase. Depending on the temperature used (eg less than about 40° C.) a suspension of the FA/MG (eg lauric acid) in water would be formed.

As will be described later, inclusion of the surfactant results in a synergistic bactericidal effect between the surfactant and the FA with respect to H. pylori elimination. It is hypothesised that use of a surfactant allows a higher concentration of FA to be present in the aqueous phase in the gastric environment and to be more able to interact with the H. pylori although the mechanism for the enhanced activity has yet to be finally determined. Why an increased effect is not observed for the MG/surfactant combination is unclear at this stage.

The FA/MG can be selected from a number of compounds which by themselves will preferably have a minimum bactericidal concentration (MBC) against H. pylori of 5 mM or less at pH 7.0. These can be selected from any one or more of capric acid, lauric acid, myristic acid, myristoleic acid, palmitoleic acid, linolenic acid, monolaurin or monomyristin for example. This list is not intended to be limiting. Reference can also be made to Table 1 of Sun et al FEMS Immun. Med. Microbiol. 36, 9-17, 2003 (the disclosure of which is included herein by way of reference). The MBC is the minimum concentration of agent(s) that result(s) in a 5 log units decrease in viable cell numbers.

The surfactant will preferably be a non-ionic surfactant derived from sorbitan esters, such as the Tweens (eg Tween 20 or Tween 80). Other options would include surfactants such as bile salt surfactants, Brij surfactants, Triton surfactants, or phospholipids or MG/FA mixtures. Again, this is not intended to be limiting. The surfactant will disperse/solubilise the FA or MG in the aqueous phase and to this extent may be seen to be acting as an emulsifying/solubilising agent. Other emulsifying/solubilising agents suitable for use in the treatment could also be used. However, the use of surfactants may also have beneficial qualities in the H. pylori treatment. Whether this is due to some form of direct action on the part of the surfactant or is due to its ability to disperse/solubilise particularly the FA in the aqueous phase while in the stomach, thus increasing the contact with the H. pylori, is unclear at this time (as discussed earlier). It is also possible that the emulsifying/surfactant properties of some of the MGs could be utilised to assist in emulsification of the FA. It is preferred that such a mixture would also include a surfactant however to take advantage of the enhanced FA bactericidal effect.

Surfactants having aggregation numbers between about 50 and about 5000 could be used in this composition. Those surfactants with lower aggregation numbers (below about 1000) are preferred. Tween 80 for example has an aggregation number of 133. The FA/MG would be more available at the micelle/aqueous solvent interface of the mixed micelles for surfactants with lower aggregation numbers than with higher. With lower aggregation numbers (eg Tween 80, 20) it is hypothesised that there would be a higher effective aqueous concentration of FA/MGs exposed to the H. pylori bacteria in the aqueous phase. However, surfactants such as a lecithin with an aggregation number of about 4000 will also be reasonably effective. While it is very much preferred to use a surfactant with a FA, it is less preferred to use a surfactant with an MG. The combination MG/surfactant will, however, still have a reasonable bactericidal effect on H. pylori.

It is also very preferable to be able to prepare a liquid dosage form that would maximise accurate and uniform dosing of the FA/MG (eg lauric acid etc). This liquid dosage form should be homogenous and uniform to ensure proper dosing and allow accurate administration of the fatty acid (eg lauric acid) and the surfactant (eg Tween 20). In addition the developed dosage form should be physically stable and fulfil the requirements of pharmaceutically acceptable suspensions such as slow sedimentation and ease of re-dispersion.

It has been found that by combining the FA/MG (eg lauric, capric, myristic acid) with a solubilising agent (eg a non-ionic surfactant) with a density modifier and a viscosity enhancer, a suitable dosage form can be prepared. This combination allows the formulation to have physical stability, ease of re-constitution on shaking, and resistance to quick phase separation. This additional inventive aspect also allows an administration form to be prepared that is capable of capturing the synergistic bactericidal effect of the FA+surfactant in particular.

The density modifier used can be selected from any one or more of anise, peppermint or fennel oils. This list is not intended to be limiting, however.

The viscosity enhancer used can be selected from any one or more of cellulose derivatives such as hydroxypropylmethyl cellulose (HPMC), carboxymethyl cellulose sodium (CMC), and other suspending agents such as xanthan gum, guar gum, sodium alginate, pectin, gelatin and starch. Again, this list is not intended to be limiting.

Preferably the density modifier is peppermint oil and the viscosity modifier is CMC. When the viscosity modifier is CMC it will preferably be included as a 2% solution (w/v). A range of 0.1 to 5% (w/v) of viscosity modifier in the solution could be used depending on the type of modifier used.

The use of density modifiers, such as peppermint oil, have the additional advantage of providing a taste-masking effect. This ameliorates the effect of the adverse taste of the lauric acid (or other FA/MGs).

The amount of density modifier in the final composition is preferably between about 0.1% and about 1.0% (v/v). The amount of the viscosity enhancer in the final composition is preferably between about 0.1% and 5.0% v/v.

The proton pump inhibitors that could be used in this invention will include lansoprazole and omeprazole. Other options would include esomeprazole, timoprazole, or picoprazole. Again, this is not intended to be limiting.

In the present invention the proton pump inhibitor needs to be made available both systemically and locally in the gastric lumen. Thus, a first dosage is administered in a form suitable to release the proton pump inhibitor systemically. Persons skilled in the art will readily appreciate appropriate administration forms to achieve this end. For example, the systemically available form could be one that is enterically coated. The requirement is that the proton pump inhibitor becomes systemically available. The systemically available proton pump inhibitor treatment is preferably given before administration of the other compounds in the treatment to allow it to take effect and be present in the patient's system when the next treatment steps are undertaken.

Solid dosage forms may be tablets, capsules, caplets, granules or pellets or the like. Semisolid forms may be gels, pastes, or the like. Liquids may be emulsions, suspensions, solutions or the like.

Preferably the proton pump inhibitor for systemic delivery is administered before fasting, for example the night before the remainder of the treatment steps. The time frames for this would be well known. One effect of this is to raise the stomach pH thus reducing the problems that can be caused by mucolytic use. Omeprazole is used medically as a proton pump inhibitor, being delivered orally, absorbed in the small intestine, and delivered systemically (by blood) to the parietal cells of the stomach where it inhibits acid secretion. This action will reduce the acidity of the stomach, and limit damage to stomach tissue by aggressive agents in the lumen (eg acid and pepsin) during the time that the mucus layer barrier is missing, disrupted or decreased in effectiveness.

Omeprazole by itself has slight bactericidal action on H. pylori (Midolo, P. D. et al 1997 JAC 39, 331-7; Jonkers D et al, 1999 JAC43, 837-9). However, the present inventors have, in particular, found that it has a powerful synergistic bactericidal effect when used in conjunction with a FA (eg lauric acid (FA C12:0)) and a suitable surfactant (eg Tween 20). It is postulated that omeprazole does this by inhibiting one or more of the proton pumping mechanisms in H. pylori. This will stress the energetics of the bacterium, aggravating the condition of the energy conserving pathways already compromised by having to expel protons and fatty acid anions at the expense of cell energy pools.

To supplement the amount of omeprazole reaching the location of colonized H. pylori under the stomach mucus layer via the systemic circulation and subsequent secretion/diffusion from the stomach mucosa, a second dose of proton pump inhibitor is introduced to the location of the H. pylori from the direction of the stomach lumen, after the mucus barrier has been disrupted by a mucolytic agent.

In current “triple therapy” prior art, proton pump inhibitors are used to decrease stomach acidity, and are not envisaged to have any direct effect on H. pylori. In any event systemic secretion of omeprazole into the lumen by stomach tissue is unlikely to produce sufficiently high concentrations of omeprazole at the site of colonisation to have any effect. Also, in the absence of suitable FA/MG concentrations, the bacteria may not be affected, since the findings of the present inventors indicate the benefit of the combination with the proton pump inhibitor is a synergistic effect and not significant with the proton pump inhibitor alone. It is this synergistic combination effect that allows the overall composition to provide such a useful alternative to existing options.

A recent paper (Olsen, J. W. & Maier, R. J. “Molecular hydrogen as an energy source for Helicobacter pylori”. Science 298, 1788-1790, 2002.) suggests that the major energy-conserving pathway in H. pylori is hydrogen uptake and oxidation by hydrogenase. If that is the case the addition of an inhibitor of bacterial hydrogenase may further enhance the benefits of the composition of the present invention to further cripple the bacterium by reducing the available energy for cell metabolism—already compromised by FA+Tween+proton pump inhibitor combination.

Thus, in the present invention the proton pump inhibitor must also be administered in a form which makes it gastrically available, such as a liquid or tablet capable of releasing the proton pump inhibitor into the stomach. To this extent, the gastrically available form and the systemically available form could be the same provided that the proton pump inhibitor is made available as needed. Clearly, while an enterically coated form may be preferred for systemic administration (as it allows release of the proton pump inhibitor into the intestine), such a form would be unsuited for use as a gastrically available form. This form is preferably administered after the mucolytic preparation has been administered. It is possible that the gastrically available form could be administered with the mucolytic but this is less preferred. The gastrically available form is preferably administered with the FA and/or MG, as is further discussed below.

It will be appreciated that whilst it is necessary to have the proton pump inhibitor available systemically and gastrically this need not necessarily require the use of two different formulations of the proton pump inhibitor, depending on the nature of the formulation. Suitable formulations for gastric/duodenal and systemic administration of suitable proton pump inhibitors will be well known to a person skilled in the formulation art.

As discussed above it is thought that there is a dual action occurring against H. pylori from the use of the two availability forms of proton pump inhibitor in the compositions and methods of the invention. The systemic form attacks the H. pylori from the patient's system and also serves to raise the gastric pH, the latter effect will minimise the potential adverse effect from exposure of the stomach tissue to damage by very low pH after the mucolytic. The gastrically available proton pump inhibitor provides sufficiently high concentrations in the stomach to have a direct and synergistic action on the H. pylori, in the presence of the FA and/or MG, once the mucus layer has been removed.

As discussed previously, one of the barriers to treating H. pylori infections is that the bacteria live beneath the layer of mucus which covers the stomach mucosa, and protects the bacterium from luminal agents. Therefore, removal or damage to the integrity of this mucus layer or disruption of its barrier effect by a suitable mucolytic preparation will expose the bacterium to the active bactericidal agents, maximising the treatment effect. The mucolytic agent N-acetylcysteine is widely used in humans in many situations, notably as a gastric mucolytic during gastroscopy. Other options will include any one or more of the suitable sulfhydryl reagents as will be well-known to a skilled person.

The use of the systemically available proton pump inhibitor (which lowers the stomach acidity) will minimise damage to the stomach lining from the effect of high levels of gastric acid and pepsin after administration of the mucolytic agent. The compositions and methods of the invention will therefore preferably allow the systemic proton pump inhibitor and mucolytic to take action followed by the fatty acid and/or MG and locally acting proton pump inhibitor. This can be achieved sequentially, individually, or grouped appropriately whether by suitable delayed release dosage forms as appropriate or otherwise.

The systemic proton pump inhibitor administration will preferably be the night before the remainder of the treatment occurs. This allows the proton pump inhibitor to take effect and also allows the stomach to empty prior to continuation of the treatment regimen. In effect this is an overnight fasting, which is important from a patient compliance perspective. Alternatively, other fasting periods could be used as desired. If there is any significant amount of lipid in the stomach the treatment will be less effective due to the propensity of the FA/MG to partition into the lipid. This will then significantly reduce the amount of FA/MG available in the aqueous phase to take action against the H. pylori (which also exists in the aqueous phase in the stomach). Thus, it is desirable for there to be a period of continued fasting (preferably for at least an hour) after administration of the FA and/or MG.

The preferable time delay between the mucolytic preparation administration and the gastric proton pump inhibitor/FA and/or MG administration should be between 15 minutes and 3 hours but could, in some circumstances, be up to about 12 hours but must be before the mucus barrier is re-established. Shorter times (20-30 minutes) are preferable to maximise patient compliance, however. The mucolytic/gastric proton pump inhibitor/FA/MG administration may be repeated for a number of days following overnight fasting. As will be readily apparent, this will preferably occur each morning following overnight fasting.

The invention in one aspect may also be seen to include the use of a proton pump inhibitor in a gastrically available form and a FA and/or MG in a form which is readily dispersed in the aqueous phase in the stomach in the preparation of a medicament for the treatment of gastric H. pylori infections in a patient who has been treated with a proton pump inhibitor in a systemically available form and a mucolytic preparation. Thus, a composition including a gastrically available proton pump inhibitor and a FA and/or MG in a suitable form for use in treating gastric H. pylori infections in a patient who has been pretreated with a systemically available proton pump inhibitor, and a mucolytic preparation may also form part of the invention. The use of such a composition in the treatment of gastric H. pylori infection in such a patient may likewise form a part of the invention. Similarly, the invention would consist of the use of a mucolytic, a gastrically available proton pump inhibitor, an FA and/or MG in a readily dispersible/soluble form in the treatment of a patient who has previously been treated with a proton pump inhibitor in a systemically available form. The composition can include a suitable carrier as would be known to the skilled person and could also include an amount of surfactant suitable to disperse/dissolve particularly the FA in the aqueous phase. Alternatively the FA could be provided in a form (such as a salt form) that more readily dissolves/disperses in the aqueous phase. As will be apparent, the provision of a pharmaceutical kit that combines the various components of the invention will also form part of the invention.

Finally, the preparation of a medicament for the treatment of H. pylori infections (and conditions resulting from this) using the various components of the present invention, also forms part of this invention.

### EXAMPLES

### Example 1

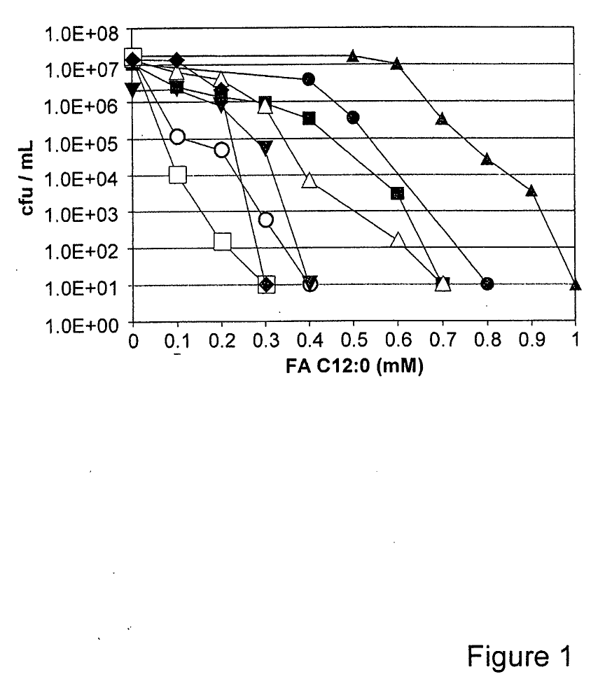

The effects of combinations of lansoprazole (0-400 μg/mL), 0.05% Tween 20, and FA C12:0 (0.1-1.0 mM) on the viability of H. pylori were examined in standard incubations. The results are shown in FIG. 1, and the MBCs for FA C12:0, with different combinations of lansoprazole and Tween 20, are summarised in Table 1.

TABLE 1
Minimal bactericidal concentrations (MBC) of FA C12:0 in media
containing different combinations of lansoprazole (LP), FA C12:0
and Tween 20, after 40 min incubation with
H. pylori at 37° C. and pH 7.4.

| | MBC |
|---|---|
| Additions to medium | (mM) |
| FA C12:0 | 1.0 |
| FA C12:0 + LP (100 μg/mL) | 0.8 |
| FA C12:0 + LP (200 μg/mL) | 0.7 |
| FA C12:0 + LP (300 μg/mL) | 0.4 |
| FA C12:0 + LP (400 μg/mL) | 0.3 |
| FA C12:0 + Tween 20 (0.05%) | 0.5 |
| FA C12:0 + Tween 20 (0.05%) + LP (100 μg/mL) | 0.4 |
| FA C12:0 + Tween 20 (0.05%) + LP (200 μg/mL) | 0.3 |

In the presence of Tween 20, increasing concentrations of lansoprazole (0, 100, 200, 300 and 400 μg/mL) in combination with selected concentrations of FA C12:0 showed profiles. in which the fatty acid (FA) became more bactericidal with increasing lansoprazole (FIG. 1, closed symbols). The MBCs calculated from these profiles reflect this trend, decreasing from 1.0 mM FA C12:0 with no lansoprazole to 0.3 mM FA C12:0 with 400 μg/mL lansoprazole (Table 1). In addition, it can be seen in FIG. 1 that, even at 400 μg/mL lansoprazole (in the absence of Tween 20), there exists a plateau region at low FA concentrations, over which H. pylori viability is hardly affected. This plateau region was eliminated in the presence of Tween.

When a similar set of experiments was performed with 100 μg/ml lansoprazole and 0.05% Tween 20 present (FIG. 1, open circles), the FA again became progressively more bactericidal in the presence of lansoprazole. The presence of the added Tween 20 made the FA even more potent than the corresponding incubation without surfactant, displacing the cell viability profile to the left (more effective killing). The corresponding MBCs decreased from 0.5 mM FA C12:0 with 0.05% Tween 20 but no lansoprazole to 0.3 mM FA C12:0 with 0.05% Tween 20 plus 200 μg/mL lansoprazole (Table 1). Importantly, the plateau region (over which low concentrations of FA had no effect, in the absence of Tween 20) disappeared. Thus, in the presence of Tween 20 there was always some effect on cell viability, even at low concentrations of FA or FA plus lansoprazole. These data in FIG. 1 and Table 1 clearly indicate that potent bactericidal synergies occur with combinations of the three molecules, FA C12:0, lansoprazole and Tween 20.

Tween 20 (polyoxyethylene sorbitan esters of about 50% lauric acid with a balance of myristic, palmitic and stearic acids) was more effective than Tween 80 (polyoxyethylene sorbitan esters of about 70% oleic acid with a balance of linoleic, palmitic and stearic acids) at decreasing the concentration of FA C12:0 required to kill H. pylori. The effect of each Tween was synergistic, as opposed to additive, since the Tween concentrations used had little effect on their own. Their probable mechanism is to increase the concentration of the FA available in aqueous solution by forming micelles. Tween 20 is more hydrophilic than Tween 80, and is more likely to enhance the FA availability. The micelle size formed may also play a part as discussed earlier. The profile of the FA concentration versus cell viability was altered by Tween surfactants, and the initial plateau range of low FA concentrations, within which no cell killing was observed, disappeared. This indicates that the FA concentration no longer needed to reach a threshold value before it began to affect cell viability.

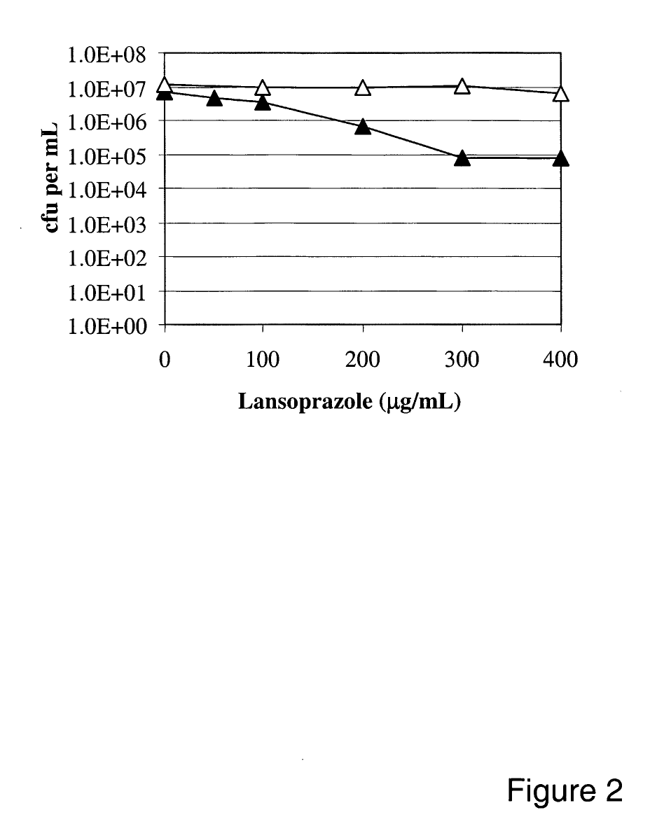

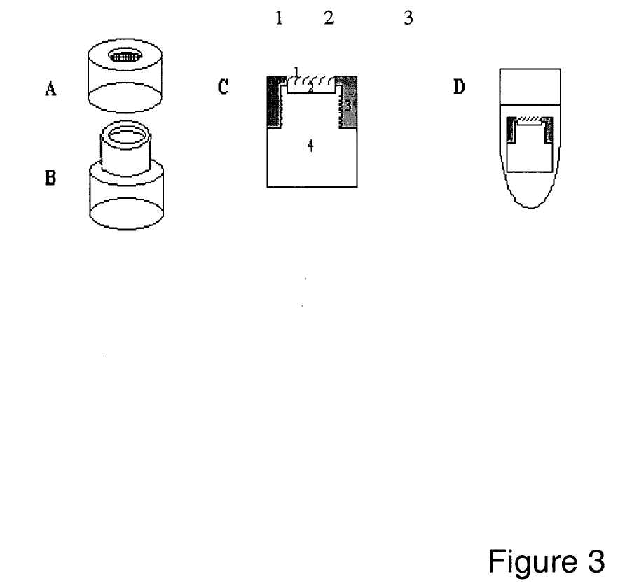

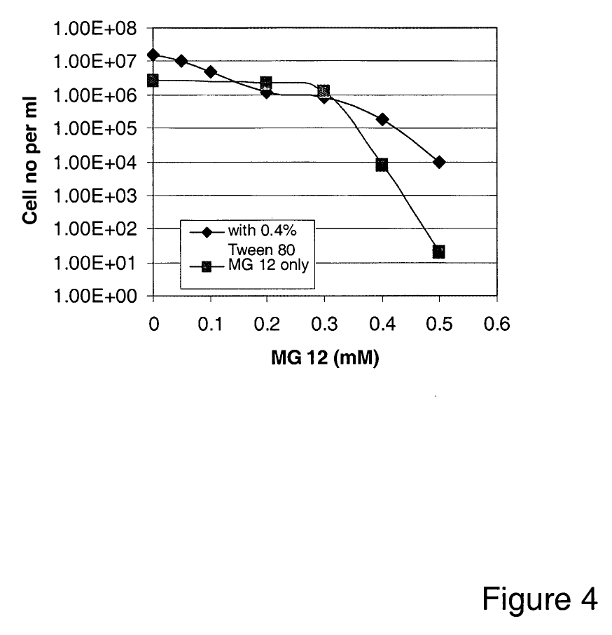

Lansoprazole is a PPI drug used to treat H. pylori infection in some triple therapies. By itself, in vitro, it has little effect on H. pylori at concentrations below 400 μg/mL (Midolo et al J. Antimicrob. Chemother. 39,331-337, (1997)). FIG. 2 confirms this observation.

At pH 7.4, the FA would have been present as its anion and is likely to form mixed micelles with the surfactant. These mixed micelles are likely to affect the concentration or activity of FA anions present at the bacterial cell surface. The FAs will also be present in the form of micelles which are very dynamic structures. Both inter- and intra-exchange between the FAs and surfactant molecules in micelles will thus be possible. Exchange between molecules in the micelles and a droplet of oil will be possible, but will occur considerably more slowly.

These data show it is possible to enhance the effectiveness of H. pylori killing by FAs, in vitro, by addition of suitable surfactant to make micelles. A triple cocktail containing surfactant, PPI and FA is more effective than any single or double mixture of these agents. It needs to be emphasised that all three ingredients can be at low concentrations which by themselves have no effect (0.3 mM FA C12:0, 0.05% Tween 20 and 200 μg/ml lansoprazole), yet together they decrease H. pylori viability by at least 6 logs in the standard incubation. This is strong supporting evidence that a synergistic effect occurs when an FA (lauric acid) is combined with a surfactant (Tween 20) to treat a bacterial infection (H. pylori).

### Example 2

In vivo, the presence of mucus between the H. pylori and the gastric lumen will interfere with access of any gastrically-available medication to the site of H. pylori infection. To test whether the presence of a mucolytic agent and mucus will affect the activity of the combination (FA+surfactant+PPI) the following in vitro test was carried out.

An H. pylori (NCTC 11637) culture was mixed with pig gastric mucus for 40 min, and then H. pylori colonies were re-isolated by dilution and plating. The re-isolated strain was used in the experiments below. PCR of the 16S rDNA piece and sequencing was used to confirm that the re-isolated bacterial strain was H. pylori.

H. pylori was grown by plating on Columbia agar (Oxoid) with 5% horse serum (Fort Richard) at 37° C. for 48 h under microaerophilic conditions (80% nitrogen, 15% carbon dioxide, 5% oxygen). Two loops of 48 h agar plate culture were inoculated into pre-warmed (37° C.) Iso-sensitest broth (Oxoid) containing 5% horse serum and incubated for 24 h under microaerophilic conditions.

This 24 h H. pylori broth culture (500 μL) was added to 3.5 g of thawed pre-warmed pig gastric mucus (previously frozen at −20° C.) and mixed. The mucus/H. pylori mixture was incubated at room temperature for 30 min under microaerophilic conditions. Aliquots of the mucus/H. pylori mixture were transferred into the test chambers (FIG. 3). The mucus/H. pylori layer was 1 mm in thickness; and after assembly the test chamber was placed in a 50 mL Falcon tube containing Iso-sensitest broth (Oxoid) with different test additives.

A diagram of the test chamber is drawn in FIG. 3.

The Falcon tube containing the mucus/H. pylori test chamber was incubated at 37° C. for 40 min under microaerophilic conditions on a rotary shaker (200 rpm). The test chamber was then removed and its surfaces rinsed three times with sterile distilled water. The mucus gel/H. pylori was removed from the test chamber and weighed. An initial 1:10 dilution of mucus was made by adding 9 volumes of Iso-sensitest broth containing 0.245 M N-acetylcysteine (NAC) and homogenizing in a glass homogeniser, to make a consistency that could be pipetted. Further 10-fold dilution series were carried out on samples in Iso-sensitest broth. Dilutions (50 μL) were then spread on Columbia agar (Oxoid) plates containing 5% horse serum, 10 mg/L vancomycin (Sigma), 330 μg/L polymixin B (Sigma), 20 mg/L bacitracin (Sigma), 10 mg/L nalidixic acid (Sigma) and 5 mg/L ampotericin B (Sigma). The plates were incubated for 4 days at 37° C. under microaerophilic conditions before counting. Final concentrations of surviving H. pylori colony forming units (cfu) were calculated per gram of mucus.

Table 2 shows the H. pylori survival in mucus, after incubation with various aqueous compositions in separate test chamber experiments carried out in parallel, as shown below. Abbreviations used are: FA=0.1 or 0.2 mM lauric acid; TW=0.05% Tween 20; OM=0.58 mM omeprazole; NAC=0.245 M N-acetylcysteine.
(a) Calculated H. pylori cfu's initially added per gram of mucus. This was obtained from a dilution series from the 24 h broth culture of H. pylori.
(b) Mucus/H. pylori in test chamber, exposed to Iso-sensitest broth for 40 min.
(c) Mucus/H. pylori in test chamber, exposed to Iso-sensitest broth containing FA for 40 min (no TW, OM or NAC).
(d) Mucus/H. pylori in test chamber, exposed to Iso-sensitest broth containing FA+TW+NAC for 40 min (no OM).
(e) Mucus/H. pylori in test chamber, exposed to Iso-sensitest broth containing FA+OM+NAC for 40 min (no TW).
(f) Mucus/H. pylori in test chamber, exposed to Iso-sensitest broth containing FA+TW+OM for 40 min (no NAC).
(g) Mucus/H. pylori in test chamber, exposed to Iso-sensitest broth containing FA+TW+OM+NAC for 40 min (complete mixture of test additives).

TABLE 2
The survival of Helicobacter pylori in mucus, incubated in the presence
of Iso-sensitest broth containing combinations of FA C12:0, Tween 20,
omeprazole, and N-Acetylcysteine.
All experiments were conducted at pH 7.0.

| | H. pylori numbers | H. pylori numbers |
|---|---|---|
| | (cfu per g mucus) | (cfu per g mucus) |
| Treatment | using 0.1 mM FA | using 0.2 mM FA |
| (a) H. pylori numbers expected, calculated | 3.06 × 10⁷ | 4.95 × 10⁶ |
| from a dilution series of the 24 h broth | | |
| (b) H. pylori numbers in mucus after 40 min | 1.29 × 10⁶ | 2.64 × 10⁴ |
| exposure to broth alone* | | |
| (c) H. pylori numbers in mucus after 40 min | 1.85 × 10⁵ | 2.59 × 10⁴ |
| exposure to broth + FA. (No TW, OM and | | |
| NAC). | | |
| (d) H. pylori numbers in mucus after 40 min | 8.08 × 10⁵ | 4.33 × 10² |
| exposure to broth + FA + TW + NAC. (No | | |
| OM). | | |
| (e) H. pylori numbers in mucus after 40 min | 4.82 × 10⁵ | <67 |
| exposure to broth + FA + OM + NAC. (No | | |
| TW). | | |
| (f) H. pylori numbers in mucus after 40 min | 5.29 × 10⁴ | 2.40 × 10³ |
| exposure to broth + FA + TW + OM. (No | | |
| NAC). | | |
| (g) H. pylori numbers in mucus after 40 min | 2.56 × 10⁴ | <67 |
| exposure to broth + FA + TW + OM + NAC | | |
| (Complete mixture of reagents). | | |

*Recovery rates were poor due to some unknown agent in mucus. The results in rows (c) to (g) should be compared with these recovery rate controls in row (b).

Analysis of Results in Example 2

Both experiments described in Example 2 show that 0.245 mM N-acetylcysteine (NAC) increases the bactericidal effect of the test additives (lauric acid, Tween 20 and omeprazole), by comparing results in sample (g) with sample (f) and all other samples. It is likely that the NAC acts by decreasing the integrity of the mucus barrier, enabling test agents to reach the H. pylori cells. The mucus after exposure to NAC appears stringy in appearance, and can be easily homogenised to a homogeneous suspension, consistent with a mucolytic change.

Omeprazole (OM) omission from the test agent mixture decreased the bactericidal effect, as shown by comparison of sample (g) with sample (d). Both samples contain mucolytic agent.

Tween 20 (TW) omission from the test agent mixture decreases the bactericidal effect, as shown by comparison of sample (g) with sample (e) in the 0.1 mM FA experiment. Both samples contain mucolytic agent.

In the 0.2 mM FA experiment, comparison of sample (c) containing FA alone as a bactericidal agent with samples (d), (e), (f) or (g) suggests that the addition of all the test additives is necessary to attain the optimal bactericidal potency. The 0.1 mM FA experiment contained a concentration of FA that on its own had minimal effect on H. pylori survival.

The results in Table 2, in which the mucus integrity has been decreased by addition of the mucolytic agent (NAC), are consistent with the results shown in FIG. 1 in which no mucus was present (and no mucolytic agent was therefore necessary). Thus the in vitro mucus/H. pylori test chamber model experiments, designed to test the effectiveness of a mucus barrier in protecting H. pylori cells from bactericidal agents, predicts that the mixture of lauric acid, Tween 20, omeprazole and N-acetylcysteine described in this invention will overcome the mucus barrier effectiveness in the stomach and kill H. pylori cells in vivo.

### Example 3

Formulation of fatty acid and surfactant dosage form.

Formulation Method
1. Weigh out the specified amounts of the fatty acid (lauric acid) and surfactant (Tween 20) in a beaker
2. Incubate in an oven at 65-70° C.
3. Incubate the specified weight of the aqueous phase at the same temperature
4. At the same temperature slowly add the oily phase to the aqueous phase
5. Homogenize at high speed for 15-30 minutes
6. Transfer into an appropriate container and label

Formulation A

| Component | Wt (g) |
|---|---|
| Lauric Acid | 9.95 |
| CMC 2% | 39.80 |
| Tween 20 | 0.25 |
| Peppermint Oil | 0.40 |

Result

Formulation A was stable on long term storage (up to 6 months). The use of the peppermint oil as the density modifier has the ability to mask the unpleasant taste of the lauric acid (a practical advantage for product use). The viscosity modifier, carboxymethyl cellulose sodium (CMC) solution (2% w/v), has the additional advantage of reducing the tendency of the lauric acid (fatty acid) to froth and foam on shaking. This is probably due to the ability of CMC to lower surface tension. It was found that if both the density enhancer and the viscosity modifier were not used, the formulation had a variety of stability problems.

The combination of the two ingredients lauric acid (a fatty acid) and a surfactant (Tween 20) on their own with an aqueous dispersion medium results in systems that are physically unstable. Such systems would not allow accurate dosing due to rapid phase separation and are therefore not suitable for administration in a liquid form. However, pharmaceutically acceptable liquid dosage forms suitable for oral administration of a combination of fatty acids/monoglycerides and surfactants can be formulated with the aid of a density modifier (eg peppermint oil) and a viscosity enhancer (eg carboxymethyl cellulose sodium).

The ability to be able to prepare a storage-stable formulation of a fatty acid or monoglyceride in a dosage form capable of integration into an aqueous system, can be seen to be an additional inventive aspect. This formulation, or combination of this formulation with other actives used in gastric therapy, may offer alternative advantages in treatment of bacterial infections for users. This would be based in part on the bactericidal effect of FA/MGs which could complement or enhance the effect of the other actives. Thus the use of the stable fatty acid/surfactant composition in the preparation of a bactericidal composition for the treatment of bacterial related infections in the gastric or duodenal tracts is an additional inventive aspect. While the formulation could be used with MGs as well, the lack of increased effect of surfactant with MG means this would be less preferred but may still be an option given the ability to provide accurate dosing.

### Example 4

Experiments to Examine Possible Interactions Between Monolaurin (MG C12) and Tween 80. The concentration profile of monolaurin on the survival of H. pylori was tested in the presence and absence of 0.4% (w/v) Tween 80, and the results are shown in FIG. 4. MG C12 on its own, has little effect in the range 0-0.3 mM. However in the range 0.3-0.5 mM MG C12 there is a sharp increase in bactericidal potency, and the killing increased by >5 log units (FIG. 4, squares). The presence of Tween 80 does not enhance the bactericidal effect of MG C12, and in fact appears to attenuate the effect slightly (FIG. 4, diamonds). Only 3 logs killing of H. pylori was observed with 0.5 mM monolaurin when 0.4% Tween 80 was present, compared with >5 logs in the absence of Tween 80.

This result shows that MGs are effective bactericidal agents. The combination with a surfactant does not provide an enhanced effect at all but, while the surfactant decreased the bactericidal effect slightly, the combination still achieved a bactericidal effect.

While in the foregoing description there has been made reference to specific components or integers of the invention having known equivalents then such equivalents are herein incorporated as if individually set forth.

Although this invention has been described by way of example only and with reference to possible embodiments thereof it is to be understood that modifications or improvements may be made without departing from the scope or spirit of the invention as defined in the attached claims.

1. A composition, in one or more parts, for the treatment of gastric and/or duodenal H. pylori infections, the composition including effective amounts of:
(a) a proton pump inhibitor in a systemically available dosage form;
(b) a mucolytic preparation;
(c) a proton pump inhibitor in a gastrically available dosage form; and
(d) a fatty acid and/or monoglyceride.

2. The composition according to claim 1 wherein the fatty acid or monoglyceride has a minimum bactericidal concentration of 5 mM or less against H. pylori.

3. The composition according to claim 1 wherein the fatty acid or monoglyceride is selected from any one or more of: capric acid, lauric acid, myristic acid, myristoleic acid, palmitoleic acid, linolenic acid, monolaurin and monomyristin.

4. The composition according to claim 1 wherein the fatty acid and/or monoglyceride are in a form capable of dispersing or dissolving in the gastric aqueous phase.

5. The composition according to claim 1 wherein the fatty acid is combined with a solubilising agent.

6-8. (canceled)

9. The composition according to claim 1 wherein the fatty acid is combined with a non-ionic surfactant, a density modifier and a viscosity enhancer.

10. The composition according to claim 1 wherein the composition comprises a fatty acid and a monoglyceride, and the monoglyceride assists in emulsifying the fatty acid.

11. The composition according to claim 1 wherein the systemically and gastrically available proton pump inhibitor are the same or different and are selected from one or more of omeprazole, lansoprazole, esomeprozole, timoprazole and picoprazole.

12. The composition according to claim 1 wherein the systemically available dosage form and/or the gastrically/duodenally available dosage form of the proton pump inhibitor is in a liquid, tablet, capsule, caplet or granule form.

13. The composition according to claim 1 wherein the systemically available dosage form of the proton pump inhibitor is an enterically coated dosage form.

14. (canceled)

15. The composition according to claim 1 wherein the gastrically available and systemically available proton pump inhibitor dosage forms are the same but are not an enterically coated dosage form.

16. The composition according to claim 1 claims wherein the mucolytic agent is selected from a suitable sulfhydryl reagent.

17. The composition according to claim 16 wherein the mucolytic agent is N-acetylcysteine.

18. The composition according to claim 1 further including at least one antibiotic.

19. A method for treating gastric and/or duodenal H. pylori infections including the steps of sequentially:
(a) administering a proton pump inhibitor in a systemically available dosage form; (b) administering a mucolytic preparation;
(c) administering a proton pump inhibitor in a gastrically and/or duodenally available form, and a fatty acid and/or a monoglyceride together or sequentially in either order; and
(d) optionally, repeating steps (b) and (c) for a time effective to treat the H. pylori infection.

20. The method according to claim 19 wherein steps (b) and (c) and (d) occur after fasting.

21. The method according to claim 19 wherein step (a) is single, unrepeated, administration step.

22. The method according to claim 19 wherein step (a) occurs before fasting.

23. The method according to claim 19 wherein step (b) occurs from about 15 minutes to about 3 hours before step (c).

24. The method according to claim 19 wherein step (c) is achieved using a single dosage form.

25-28. (canceled)

29. A composition including a fatty acid together with a surfactant, viscosity enhancer and a density modifier.

30. The composition according to claim 29 wherein the fatty acid is capric, lauric, or myristic acid.

31. The composition according to claim 29 wherein the surfactant has an aggregation number of between about 50 and about 5000.

32. The composition according to claim 31 wherein the aggregation number is less than about 1000.

33. The composition according to claim 29 wherein the surfactant is Tween 20 or Tween 80.

34. The composition according to claim 29 wherein the viscosity enhancer comprises one or more cellulose derivatives selected from the group consisting of hydroxypropylmethyl cellulose (HPMC), carboxymethyl cellulose sodium (CMC), and suspending agents.

35. The composition according to claim 29 wherein the density modifier is selected from any one or more of anise, peppermint and fennel oils.

36. A pharmaceutical kit, the kit including the components (a) to (d) of the composition according to claim 1.

37. The kit according to claim 36 wherein component (d) is combined with a non-ionic surfactant.

38. The kit according to claim 37 wherein (d) is combined with a density modifier and a viscosity enhancer.

39. The use of the composition of claim 1, in the management of any disease state caused, or believed to be caused by, H. pylori.

40. The use of the method of claim 19 in the management of any disease state caused, or believed to be caused by, H. pylori.

41. The method according to claim 19 wherein the systemically and gastrically available proton pump inhibitor are the same or different and are selected from one or more of omeprazole, lansoprazole, esomeprozole, timoprazole or picoprazole and wherein the fatty acid or monoglyceride is selected from any one or more of: capric acid, lauric acid, myristic acid, myristoleic acid, palmitoleic acid, linolenic acid, monolaurin and monomyristin.

42. The use of the kit of claim 37 in the management of any disease state caused, or believed to be caused by, H. pylori.

43. The composition according to claim 34, wherein the suspending agent is selected from the group consisting of xanthan gum, guar gum, sodium alginate, pectin, gelatin and starch.
